(19) United States
(12) Patent Application Publication
(10) Pub. No.: US 2012/0114589 A1
(43) Pub. Date: May 10, 2012

(54) SWELLABLE BIODEGRADABLE POLYMERIC MATRICES AND METHODS

(76) Inventors: Emily R. Rolfes-Meyering, Eden Prairie, MN (US); Stephen Chudzik, St. Paul, MN (US); Pamela J. Reed, Minnetonka, MN (US)

(21) Appl. No.: 13/125,521

(22) PCT Filed: Oct. 22, 2009
(86) PCT No.: PCT/US09/05739

§ 371 Date: Nov. 28, 2011

Related U.S. Application Data
(60) Provisional application No. 61/197,006, filed on Oct. 22, 2008.

(51) Int. Cl. A61L 31/06; A61P 41/00; C08B 30/18

(52) U.S. Cl. 424/78.3; 525/54.31; 523/113

(57) ABSTRACT

The invention provides biodegradable polymeric hydrogel matrices having excellent durability and swellability. The matrices are formed from a combination of poly-α(1→4)glucopyranose macromer and a biocompatible biostable hydrophilic macromer. The matrices can be used in association with a medical device or alone. In some methods the polymeric matrix is placed or formed at a target site in which the matrix swells and occludes the target area, and is able to be degraded at the target site after period of time.

### CROSS-REFERENCE TO RELATED APPLICATION

The present non-provisional patent Application claims priority under 35 U.S.C. §119(e) from U.S. Provisional Patent Application having Ser. No. 61/197,006, filed on Oct. 22, 2008, and titled SWELLABLE BIODEGRADABLE POLYMERIC MATRICES AND METHODS, wherein the entirety of said provisional patent application is incorporated herein by reference.

### FIELD OF THE INVENTION

The invention is directed to biodegradable hydrogels, and compositions and methods for their preparation. The invention also relates to systems and methods for the occlusion of an internal portion of the body by an implanted or formed article.

### BACKGROUND

A hydrogel is typically thought of as an insoluble matrix of crosslinked hydrophilic polymers having the capacity to absorb large amounts of water. Due to their physical properties and ability to be prepared from biocompatible materials, hydrogels have considerable use in biomedical applications. For example, hydrogels have been used as material for the treatment of wounds, as well as vehicles for the release of drugs. Hydrogels have also been used as coatings on the surface of medical devices, and can be used to improve the hydrophilicity or lubricity of a medical device surface.

Hydrogels are typically characterized by their capacity to swell upon absorption of water from a dehydrated state. This swelling can be affected by conditions in which the hydrogel is placed, such as by pH, temperature, and the local ion concentration and type. Several parameters can be used to define or characterize hydrogels in a swollen state, including the swelling ratio under changing conditions, the permeability coefficient of certain solutes, and the mechanical behavior of the hydrogel under conditions of its intended use.

Hydrogels that undergo a considerable degree of swelling can be useful for many medical applications in the body in which the hydrogel is placed, or is formed. However, hydrogels having a high degree of swelling may also be structurally unsuitable for use in the body. For example, considerable swelling may cause the hydrogel to become fragile, and fracture or fragment upon contact with body tissue. This could cause the hydrogel, or a device associated with the hydrogel, to lose its functionality, or could introduce complications in the body if a portion of the hydrogel is dislodged from the target site.

### SUMMARY

The present invention provides swellable biodegradable polymeric matrices, and systems for forming these matrices. The invention also provides methods for treating a target site in the body using these biodegradable matrices. The swellable biodegradable polymeric matrices are formed from a composition including a biocompatible biodegradable α(1→4)glucopyranose polysaccharide macromer and a biocompatible biostable macromer. The inclusion of these two matrix-forming components provides the polymeric matrix with a particular crosslinked architecture having excellent swellability and durability, while also being degradable after a period of time in the body.

The matrices are swellable, or can be provided in a swollen state. For example, the matrices can be placed at the target site in a swollen form or placed in a dried or partially dried form after which they rehydrate and swell. Alternatively, the matrices are formed by in situ polymerization of the macromer components at the target site. During the course of rehydration, the swelling does not cause structural defects in the matrix. The use of the swellable polymeric matrices as described herein can therefore provide improved function in vivo. For example, the polymeric matrices are less likely to fracture following swelling. This can provide better occlusion or blockage at the target area. The swollen matrix is durable for a period of time before substantial degradation of the matrix occurs.

The polymeric matrices of the invention provide many advantages for treatment of a target location in the body. Unlike matrices formed from other polysaccharides, the matrices formed from the α(1→4)glucopyranose macromer are degradable in the presence of an amylase-containing fluid, such as body fluid. The matrices can be placed or formed at a target portion of the body and then left there to treat the target site, following which they can be degraded. Therefore, there is not a need to explant the matrix from the subject. Further, the degradation products are not harmful to the host and can be metabolized or excreted from the body.

In one aspect, the invention provides a matrix-forming composition. The composition includes poly-α(1→4)glucopyranose comprising pendent polymerizable groups, and a biocompatible biostable hydrophilic polymer comprising a pendent polymerizable group. The composition can be used in a method for forming a biocompatible biodegradable swellable or swollen polymeric matrix wherein the polymerizable groups are activated to cause crosslinking of the poly-α(1→4)glucopyranose and the biocompatible biostable hydrophilic polymer, thereby forming the biodegradable matrix. Exemplary polymerization initiators that can be used in the composition include photo-initiators and redox initiators.

In another aspect, the invention provides a swellable or swollen biodegradable polymeric matrix formed of a crosslinked network of polymeric material comprising first and second polymer-containing segments. The first polymer-containing segment of the crosslinked network comprises poly-α(1→4)glucopyranose, and the second polymer-containing segment comprises a biocompatible biostable hydrophilic polymer. In a partially or fully dehydrated form, the matrix is substantially swellable and provides a durable hydrogel that is degradable after a period of time in the body.

In some aspects the ratio (weight) between the α(1→4)glucopyranose and biocompatible biostable hydrophilic polymer is in the range of about 200:1 to about 1:10, about 50:1 to about 1:5, or about 10:1 to about 1:2.

In some aspects the biostable polymer used to form the second polymer-containing segment comprises an oxyalkylene polymer, such as an alkylene oxide polymer. Exemplary alkylene oxide polymers include poly(propylene glycol) (PPG) and poly(ethylene glycol) (PEG).

In some formations, the polymeric matrix is capable of swelling in water to a weight of about 1.5 times its weight or greater in a dehydrated form. In some formations the matrix is capable of exerting a swelling force of about 100 g/cm² or greater from a dehydrated form. In some formations, the polymeric matrix is capable of swelling in water to a size of at least about 25% greater than its size in a dehydrated form.

The polymeric matrices can be used alone at the target area, or can be used in association with a device. For example, in some aspects, the polymeric matrices can be in the form of an overmold, or in the form of a coating on an implantable medical device. The non-hydrogel portion of the device can facilitate delivery and function of the degradable matrix at the target site.

The degradable matrices can be used in a medical procedure in a swollen state to occlude, seal, block, or space fill a target area of the body, and provide a desired biological effect at the site. Generally, the method includes a step of implanting an article comprising a swellable polymeric matrix according to the invention, or forming a matrix at a target location in the body. For example, the swellable polymeric matrices can be delivered to the target area in a dry, or partially dry (dehydrated) state, where at the target area, the matrices become hydrated and swell to occlude, block, fill, or seal the area, or the like. The occlusion or blockage can prevent the movement of biological fluids, tissue, or other biological material, across or into the occluded area.

### BRIEF DESCRIPTION OF THE DRAWINGS

FIG. 1 is a graph showing degradation of maltodextrin-poly(ethylene glycol) filaments in control and amylase solutions over time.

FIG. 2 is a graph showing degradation of maltodextrin-poly(ethylene glycol) filaments in amylase solutions over time.

### DETAILED DESCRIPTION

The embodiments of the present invention described below are not intended to be exhaustive or to limit the invention to the precise forms disclosed in the following detailed description. Rather, the embodiments are chosen and described so that others skilled in the art can appreciate and understand the principles and practices of the present invention.

All publications and patents mentioned herein are hereby incorporated by reference. The publications and patents disclosed herein are provided solely for their disclosure. Nothing herein is to be construed as an admission that the inventors are not entitled to antedate any publication and/or patent, including any publication and/or patent cited herein.

The present invention provides biocompatible biodegradable polymeric matrices that can be used in the body to provide a medical effect. These polymeric matrices can be provided to the target site in a pre-formed state that is either partially or fully dehydrated, or can be provided in a swollen state. If dehydrated, the matrices can swell at the target site. The polymeric matrices can also be formed in situ at a target site, by polymerization of a composition containing matrix-forming materials at the target location.

Typically, the swollen hydrogel is left at the target site for a desired period of time. This period of time is generally sufficient to provide treatment at the site. For example, the swollen hydrogen may block or occlude the target site. After a period of time in the body, the hydrogel becomes degraded. The degradation can be caused entirely or partially by amylases causing the enzymatic degradation of the poly α(1→4)glucopyranose segments. Enzymatic degradation of the poly α(1→4)glucopyranose also results in liberation of the polymeric segments formed from the biocompatible hydrophilic polymers, which can be eliminated from the body. Serum concentrations for amylase are estimated to be in the range of about 50-100 U per liter.

Depending on the linkage of the polymerized groups to the polymeric segments, non-enzymatic hydrolysis of unsaturated esters may also occur, further promoting the degradation of the matrix.

The swellable biodegradable polymeric matrix can be used in various forms. In some aspects, the matrix is used by itself (i.e., without being associated with a non-matrix article). In other aspects, the biodegradable polymeric matrix is used in association with an implantable medical article. For example, in some modes of practice the matrix can be in a form of an overmold on a medical device. The matrix can also be in the form of a coating on a medical device.

One component (i.e., the first component) that is used to form the swellable biodegradable polymeric matrix is a α(1→4)glucopyranose polymer comprising pendent polymerizable groups. Another component (i.e., the second component) is a biocompatible hydrophilic polymer that comprises a pendent polymerizable group. The polymerizable groups on these polymeric reagents can be reacted to form a polymeric matrix that is swellable to a durable hydrogel, but that is also degradable in vivo after a period of time in the body.

A “swellable polymeric matrix” refers to a crosslinked matrix of polymeric material formed from at least the first and second components. The polymeric matrix can either be dehydrated or can contain an amount of water that is less than the amount of water present in a fully swollen matrix (the fully hydrated matrix being referred to herein as a “hydrogel” or “swollen matrix”). The invention contemplates the matrix in various levels of hydration. In some modes of practice, the matrix is not fully hydrated when delivered to a target site in the body for occlusion.

Polymerizable groups are reactive groups pendent from the polymers that form the swellable biodegradable polymeric matrix. The polymeric components used to form the biodegradable matrix (i.e., macromers) includes one or more “polymerizable group(s)” which generally refers to a chemical group that is polymerizable in the polymerizable groups presence of free radicals. Polymerizable groups generally include a carbon-carbon double bond that can be an ethylenically unsaturated group or a vinyl group. Exemplary include acrylate groups, methacrylate groups, ethacrylate groups, 2-phenyl acrylate groups, acrylamide groups, methacrylamide groups, itaconate groups, and styrene groups.

Polymers can be effectively derivatized in organic, polar, or anhydrous solvents, or solvent combinations to produce macromers. Generally, a solvent system is used that allows for polymer solubility and control over the derivatization with polymerizable groups. Polymerizable groups such as glycidyl acrylate can be added to polymers (including polysaccharides) in straightforward synthetic processes. In some aspects, the polymerizable group is present on the macromer at a molar ratio of 0.05 μmol or greater of polymerizable group (such as an acrylate group) per 1 mg of macromer.

Polymerizable groups can be “pendent” from the macromer at more than one location along the polymer backbone. In some cases the polymerizable groups are randomly located along the length of the polymer backbone. Such randomly spacing typically occurs when the macromer is prepared from a polymer having reactive groups along the length of the polymer, and the polymer is reacted with a limited molar quantity of a compound having the polymerizable group. For example, polysaccharides described herein have hydroxyl groups along the length of the polysaccharide, and a portion of these hydroxyl groups are reacted with a compound having a hydroxyl-reactive group and a polymerizable group.

In other cases one or more polymerizable groups are pendent from the macromer at one or more defined locations along the polymer backbone. For example, a polymer used for the synthesis of the macromer can have a reactive group at its terminus, or reactive groups at its termini. Many polymers prepared from monomers with reactive oxygen-containing groups (such as oxides) have hydroxyl-containing terminal ends that can be reacted with a compound having a hydroxyl-reactive group and a polymerizable group to provide the macromer with polymerizable groups at its termini.

The macromers of the invention are based on biocompatible polymers. The term “biocompatible” (which also can be referred to as “tissue compatible”) generally refers to the inability of a component, composition, or article to promote a measurably adverse biological response in the body. A biocompatible component, composition, or article can have one or more of the following properties: non-toxic, non-mutagenic, non-allergenic, non-carcinogenic, and/or non-irritating. A biocompatible component, composition, or article, in the least, can be innocuous and tolerated by the body. A biocompatible component, by itself, may also improve one or more functions in the body.

In the context of the present invention, the inventive swellable biodegradable polymeric matrices can be shown to be biocompatible in one or more ways. For example, the matrix-forming compositions can be biocompatible and do not include a component (or an amount of a component) that adversely affects tissue, such as a component that is that is toxic to cells.

Polymers and macromers used for making the swellable biodegradable polymeric matrices of the invention can be described in terms of molecular weight. “Molecular weight,” as used herein, more specifically refers to the “weight average molecular weight” or M_{w}, which is an absolute method of measuring molecular weight and is particularly useful for measuring the molecular weight of a polymer (preparation), such as macromer preparations. Polymer preparations typically include polymers that individually have minor variations in molecular weight. In some cases, the polymers have a relatively higher molecular weight (e.g., versus smaller organic compounds) and such minor variations within the polymer preparation do not affect the overall properties of the polymer preparation. The weight average molecular weight (M_{w}) can be defined by the following formula:

$${M}_{w} = \frac{{\sum }_{i} {N}_{i} {M}_{i}^{2}}{{\sum }_{i} {N}_{i} {M}_{i}}$$

wherein N represents the number of moles of a polymer in the sample with a mass of M, and Σᵢ is the sum of all NᵢMᵢ (species) in a preparation. The M_{w} can be measured using common techniques, such as light scattering or ultracentrifugation. Discussion of M_{w} and other terms used to define the molecular weight of polymer preparations can be found in, for example, Allcock, H. R. and Lampe, F. W., Contemporary Polymer Chemistry; pg 271 (1990).

The swellable biodegradable polymeric matrices of the invention are prepared using a poly-α(1→4)glucopyranose-based macromer. A α(1→4)glucopyranose polymer includes repeating glucopyranose monomeric units having α(1→4) linkages that are capable of being enzymatically degraded. Exemplary α(1→4)glucopyranose polymers include maltodextrin, amylose, cyclodextrin, and polyalditol. Maltodextrins generally refer to those polymer preparations having a lower molecular weight than amylose preparations. Cyclodextrins are low molecular weight cyclic α(1→4)glucopyranose polymers.

Maltodextrin is typically generated by hydrolyzing a starch slurry with heat-stable α-amylase at temperatures at 85-90° C. until the desired degree of hydrolysis is reached and then inactivating the α-amylase by a second heat treatment. The maltodextrin can be purified by filtration and then spray dried to a final product. Maltodextrins are typically characterized by their dextrose equivalent (DE) value, which is related to the degree of hydrolysis defined as: DE=MW dextrose/number-averaged MW starch hydrolysate×100. Generally, maltodextrins are considered to have molecular weights that are less than amylose molecules.

A starch preparation that has been totally hydrolyzed to dextrose (glucose) has a DE of 100, whereas starch has a DE of about zero. A DE of greater than 0 but less than 100 characterizes the mean-average molecular weight of a starch hydrolysate, and maltodextrins are considered to have a DE of less than 20. Maltodextrins of various molecular weights are commercially available.

As used herein, “amylose” or “amylose polymer” refers to a linear polymer having repeating glucopyranose units that are joined by α-1,4 linkages. Some amylose polymers can have a very small amount of branching via α-1,6 linkages (about less than 0.5% of the linkages) but still demonstrate the same physical properties as linear (unbranched) amylose polymers do. Generally amylose polymers derived from plant sources have molecular weights of about 1×10⁶ Da or less. Amylopectin, comparatively, is a branched polymer having repeating glucopyranose units that are joined by α-1,4 linkages to form linear portions and the linear portions are linked together via α-1,6 linkages. The branch point linkages are generally greater than 1% of the total linkages and typically 4%-5% of the total linkages. Generally amylopectin derived from plant sources have molecular weights of 1×10⁷ Da or greater.

Exemplary maltodextrin and amylose polymers have molecular weights ranging from about 500 Da to about 500,000 Da, about 1000 Da to about 300,000 Da, and about 5000 Da to about 100,000 Da.

Maltodextrin and amylose polymers of various molecular weights are commercially available from a number of different sources. For example, Glucidex™ 6 (ave. mw ˜95,000 Da) and Glucidex™ 2 (ave. mw ˜300,000 Da) are available from Roquette (France); and MALTRIN™ maltodextrins of various molecular weight, including molecular weights from about 12,000 Da to 15,000 Da, are available from GPC (Muscatine, Iowa).

The decision of using amylose of a particular size range may depend on factors such as the physical characteristics of the composition (e.g., viscosity), the desired rate of degradation of the swellable matrix formed, and the presence of other optional components in the matrix, such as bioactive agents.

A non-reducing polysaccharide can also be used as degradable polymeric material for forming swellable biodegradable matrices. An exemplary non-reducing polysaccharide comprises polyalditol which is available from GPC (Muscatine, Iowa).

Refinement of the molecular weight of a polymer preparation (such as polysaccharide preparations) can be carried out using diafiltration. Diafiltration of polysaccharides such as maltodextrin can use ultrafiltration membranes with differing pore sizes. As an example, use of one or more cassettes with molecular weight cut-off membranes in the range of about 1K to about 500 K can be used in a diafiltration process to provide polysaccharide preparations with average molecular weights in the range of less than 500 K Da, in the range of about 5 K Da to about 30 K Da, in the range of about 5 K Da to about 30 K Da, in the range of about 10 K Da to about 30 K Da, or in the range of about 1 K Da to about 10 K Da.

Modification of a α(1→4)glucopyranose polymer, such as amylose or maltodextrin, to provide pendent polymerizable groups can be carried out using known techniques. In some modes of preparation, a portion of the hydroxyl groups (which are naturally pendent from α(1→4)glucopyranose polymer) are reacted with a compound having a hydroxyl-reactive group and a polymerizable group. For example, commonly assigned patent application, published as U.S. Pub No. 2007/0065481 (Chudzik et al.) describes modification of α(1→4)glucopyranose polymers to provide pendent acrylate and methacrylate groups.

Modification of α(1→4)glucopyranose polymers with polymerizable groups is explained with reference to the following structure. For example, a portion of the α(1→4)glucopyranose with a pendent polymerizable group can have the following structure:

[M]-[L]-[X]

wherein M is a monomeric unit of the α(1→4)glucopyranose polymer, and in the pendent chemical group ([L]-[X]), X is the unsaturated polymerizable group, and L is a chemical group linking the unsaturated polymerizable group to the glucopyranose monomeric unit.

In some cases the chemical linking group L includes a cleavable ester bond. A compounds having a polymerizable group and a hydroxyl reactive groups such as acetal, carboxyl, anhydride, acid halide, and the like, can be used to form a hydrolytically cleavable covalent bond between the polymerizable group and the α(1→4)glucopyranose backbone. For example, the method can provide a α(1→4)glucopyranose polymer with a pendent group having a polymerizable group, the polymerizable group linked to the polysaccharide backbone via a chemical moiety including a cleavable ester bond. In these aspects, the swellable biodegradable polymeric matrix will include a polymeric matrix having enzymatically degradable α(1→4)glucopyranose polymer segments and non-enzymatically hydrolytically cleavable chemical linkages between the degradable α(1→4)glucopyranose polymer segments.

Other cleavable chemical linkages that can be used to bond the polymerizable group to the α(1→4)glucopyranose polymer include peroxyester groups, disulfide groups, and hydrazone groups.

In some cases the hydroxyl reactive groups include those such as isocyanate and epoxy. These groups can be used to form a non-cleavable covalent bond between the pendent polymerizable group and the polysaccharide backbone. In these aspects, the swellable biodegradable polymeric matrix will include a polymeric matrix having enzymatically degradable α(1→4)glucopyranose polymer segments and non-cleavable chemical linkages between the monomeric units of the α(1→4)glucopyranose polymer and the unsaturated polymerizable groups.

Exemplary synthetic processes use a α(1→4)glucopyranose polymer (such as maltodextrin) dissolved in dimethylsulfoxide and reacted with a compound having a methacrylate or acrylate group and a hydroxyl reactive group selected from carboxylate, acid chloride, anhydride, azido, and cyanato. Exemplary compounds include (acryloyloxy)propanoic acid, 3-chloro-3-oxopropyl acrylate, 3-azido-3-oxopropyl acrylate, 2-isocyanatoethyl acrylate, methacrylic anhydride, methacrylic acid, and acrylic acid.

The α(1→4)glucopyranose polymer can be prepared with a desired number of pendent polymerizable (e.g., acrylate, methacrylate, etc.) groups suitable for formation of the swellable biodegradable polymeric matrices of the present invention. For example, levels of acrylation or methacrylation can be carried out by controlling the amount of reactive compound to the amount of α(1→4)glucopyranose polymer in the reaction mixture. In some aspects, the polymerizable group is present on the α(1→4)glucopyranose macromer at a molar quantity of 0.05 mmol or greater of polymerizable group (such as an acrylate group) per 1 gram of polymer (measurements can also be expressed in μmol/mg). In some aspects the α(1→4)glucopyranose is derivatized with polymerizable groups in amount in the range from about 0.05 mmol to about 0.7 mmol of polymerizable group per 1 gram of polymer. In some favored modes of practice, the biocompatible biodegradable matrices are formed using a α(1→4)glucopyranose polymer having a level of polymerizable group derivatization in the range of about 0.1 mmol to about 0.4 mmol polymerizable group (e.g., acrylate, methacrylate) per gram of polysaccharide.

The α(1→4)glucopyranose polymer may also include one or more other chemical modifications that are different than those provided by the polymerizable group. The α(1→4)glucopyranose polymer may be modified in a way to change its chemical properties. Generally, if such modifications are made they do not adversely impact the ability of the α(1→4)glucopyranose to be useful in forming a biodegradable matrix.

For example, the α(1→4)glucopyranose polymer may be derivatized with hydrophobic groups. This may be useful in reducing the hydrophilic character of the matrix and in turn could decrease the rate that the matrix degrades when in contact with tissue or body fluid. Hydrophobic groups can be added to the polysaccharide in a manner similar to that of adding polymerizable groups. For example a compound having a hydrophobic group, such as an alkyl chain of a fatty acid, and a group reactive with a hydroxyl group of the polysaccharide is used to derivatize the α(1→4)glucopyranose polymer. Exemplary compounds and methods for adding hydrophobic groups are described in U.S. Pub No. 2007/0065481 (supra), see Example 46. If hydrophobic groups are added, the derivatized α(1→4)glucopyranose polymer desirably remains soluble in a biocompatible application composition and is able to be polymerized into a swellable biodegradable polymeric matrix.

The α(1→4)glucopyranose-based macromer can be used at a final concentration in the matrix-forming composition sufficient to provide a matrix that can be swollen to a durable hydrogel matrix that is capable of degrading over a period of time in the body.

In many modes of practice the α(1→4)glucopyranose-based macromer is present in the composition used to form the swellable biodegradable at a concentration in the range of about 50 mg/mL to about 600 mg/mL, about 100 mg/mL to about 500 mg/mL, about 150 mg/mL to about 450 mg/mL, or about 200 mg/mL to about 400 mg/mL. In some modes of practice, the α(1→4)glucopyranose-based macromer is used in an amount of about 300 mg/mL

These concentration ranges represent the total amount of α(1→4)glucopyranose-based macromer is present in the composition (the composition also including the second macromer). It should be recognized that in some modes of practice, the swellable biodegradable polymeric matrices are formed by redox polymerization, in which two independent liquid compositions are mixed, and upon mixing the macromers immediately polymerize to form the matrix. Given this, prior to mixing, the α(1→4)glucopyranose-based macromer may be present in one of the compositions in an amount that is greater than the ranges described above (such as twice the concentration), but upon mixing the α(1→4)glucopyranose-based macromer (prior to being incorporated into the matrix by polymerization) has a concentration within the ranges described above.

In addition to the α(1→4)glucopyranose-based macromer, compositions for making the swellable biodegradable polymeric matrices also include a hydrophilic biocompatible macromer comprising one or more pendent polymerizable groups. For purposes of discussion, this additional macromer is referred to herein as the “second macromer.” A biostable biocompatible polymer refers to one that does not break down into monomeric units when placed in contact with tissue according to the methods of the invention, but yet is biocompatible and does not cause adverse affects in the body. Such a biostable biocompatible polymer may be eliminated from the body through urination or excretion.

Optionally, other materials, such as other macromers, can be used to form the swellable biodegradable polymeric matrix. These may be referred to as “third macromer,” “fourth macromer,” etc. If any additional macromers are used to form the matrix, these may be biodegradable, or biostable.

Exemplary polymers that that can be used to form the second macromer can be based on one or more of the following hydrophilic biocompatible polymers: poly(vinylpyrrolidone) (PVP), poly(ethylene oxide) (PEO), poly(ethyloxazoline), poly(propylene oxide) (PPO), poly(meth)acrylamide (PAA) and poly(meth)acrylic acid, poly(ethylene glycol) (PEG) (see, for example, U.S. Pat. Nos. 5,410,016, 5,626,863, 5,252,714, 5,739,208 and 5,672,662) PEG-PPO (copolymers of polyethylene glycol and polypropylene oxide), hydrophilic segmented urethanes (see, for example, U.S. Pat. Nos. 5,100,992 and 6,784,273), and polyvinyl alcohol (see, for example, U.S. Pat. Nos. 6,676,971 and 6,710,126).

In some aspects, the second macromer used to form the biodegradable matrix has a molecular weight in the range of 100 Da to about 40 kDa, or about 200 Da to about 20 kDa, or about 200 Da to about 10 kDa, or about 200 Da to about 5 kDa, or about 200 Da to about 2,500 Da.

In some aspects, the second macromer is formed from an oxyalkylene polymer. An oxyalkylene polymer refers to a polymer that includes repeating units of the formula component —(R¹—O)—, where R¹ is a substituted or unsubstituted divalent hydrocarbon group having 1 to about 8 carbon atoms. In some modes of practice, R¹ is a hydrocarbon group having 2, 3, or 4 carbon atoms. The oxyalkylene polymer can be formed from monomeric units in which R¹ is different. For example, the oxyalkylene polymer can be formed from a combination of monomeric units wherein R¹ individually, has 2 carbon atoms and 3 carbon atoms.

The oxyalkylene polymer can also be formed from monomeric units other than —(R¹—O)—. In some modes of practice the monomeric units of —(R¹—O)— in the oxyalkylene polymer account for 50 weight percent of the polymer or greater.

The oxyalkylene polymer can be an alkylene oxide polymer such as an ethylene glycol or propylene glycol polymer (e.g., poly(ethylene glycol) and poly(propylene glycol), respectively). In some cases an ethylene glycol polymer or oligomer having the structure HO—(CH₂—CH₂—O)ₙ—H is used to form the second macromer for the biodegradable matrix. As an example, the value of n ranges from about 3 to about 150 and the number average molecular weight (Mn) of the poly(ethylene glycol) ranges from about 100 Da to about 5000 Da, more typically ranging from about 200 Da to about 3500 Da, from about 250 Da to about 2000 Da, from about 250 Da to about 1500 Da, or about 400 Da to about 1000 Da.

Polyetherester copolymers can also be used to form the second macromer. Generally speaking, polyetherester copolymers are amphiphilic block copolymers that include hydrophilic (for example, a polyalkylene glycol, such as polyethylene glycol(PEG)) and hydrophobic blocks (for example, polyethylene terephthalate). Examples of block copolymers include poly(ethylene glycol)-based and poly(butylene terephthalate)-based blocks (PEG/PBT polymer). Examples of these types of multiblock copolymers are described in, for example, U.S. Pat. No. 5,980,948. PEG/PBT polymers are commercially available from Octoplus BV (Leiden, Netherlands), under the trade designation PolyActive™

Other PEG-containing block copolymers, such as those including one or more polymeric blocks selected from poly(hydroxybutyrate) (PHB), poly(oxyethylene) (POE), poly(caprolactone) (PCL), and poly(lactide) (PLA) are available from Advanced Polymer Materials, Inc. (Lachine, QC, Canada).

Many polymers prepared from monomers with reactive oxygen-containing groups (such as oxides) have hydroxyl-containing terminal ends which can be reacted with a compound having a hydroxyl-reactive group and a polymerizable group to provide the macromer with polymerizable groups at its termini.

In some aspects the swellable degradable polymer matrix is formed from at least a α(1→4)glucopyranose-based macromer and linear oxyalkylene macromer. Linear oxyalkylene polymers are described herein. In some aspects the swellable degradable polymer matrix is formed from at least an α(1→4)glucopyranose-based macromer and a branched compound containing oxyalkylene polymeric portions. In some aspects the swellable degradable polymer matrix is formed from at least an α(1→4)glucopyranose-based macromer and a second macromer that based on a linear hydrophilic biocompatible polymer, and a third macromer that is based on a branched compound comprising hydrophilic biocompatible polymer arms. Branched compounds containing oxyalkylene polymeric portions are described herein.

An oxyalkylene polymer can be effectively derivatized to add polymerizable groups to produce oxyalkylene based macromers. Polymerizable groups such as glycidyl acrylate, glycidyl methacrylate, acrylic or methacrylic acid can be reacted with the terminal hydroxyl groups of these polymers to provide terminal polymerizable groups. Acrylate and methacrylate-containing poly(ethylene glycols) or poly(propylene glycols) are also commercially available (for example from Aldrich Chemicals). Exemplary levels of derivation are in the range of about 0.001 mol to about 0.01 mol polymerizable group per gram of oxyalkylene polymer.

Some specific examples of alkylene oxide polymer-based macromers include, poly(propylene glycol)₅₄₀-diacrylate, poly(propylene glycol)₄₇₅-dimethacrylate, poly(propylene glycol)₉₀₀-diacrylate, include poly(ethylene glycol)₂₅₀-diacrylate, poly(ethylene glycol)₅₇₅-diacrylate, poly(ethylene glycol)₅₅₀-dimethacrylate, poly(ethylene glycol)₇₅₀-dimethacrylate, poly(ethylene glycol)₇₀₀-diacrylate, and poly(ethylene glycol)₁₀₀₀-diacrylate.

A “non-linear” or “branched” compound having polymeric portions refers to those having a structure different than a linear polymer (which is a polymer in which the molecules form long chains without branches or cross-linked structures). Such a compound can have multiple polymeric “arms” which are attached to a common linking portion of the compound. Non-linear or branched compounds are exemplified by, but not limited to, those having the following general structures:

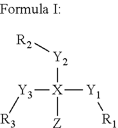

wherein X is a linking atom, such as one selected from C or S, or a linking structure, such a homo- or heterocyclic ring; Y₁ to Y₃ are bridging groups, which can independently be, for example, —Cₙ—O—, wherein n is 0 or an integer of 1 or greater; R₁ to R₃ are independently hydrophilic polymeric portions, which can be the same or different, and have one or more pendent polymerizable groups; and Z is a non-polymeric group, such as a short chain alkyl group;

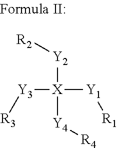

wherein X is a linking atom, such as one selected from C or S, or a linking structure, such a homo- or heterocyclic ring; Y₁ to Y₄ are bridging groups, which can individually be, for example, —Cₙ—O—, wherein n is 0 or an integer of 1 or greater; and R₁ to R₄ independently hydrophilic polymeric portions, which can be the same or different, and have one or more pendent polymerizable groups; and

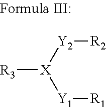

wherein X is a linking atom or group, such as one selected from N, C—H, or S—H, or a linking structure, such a homo- or heterocyclic ring; Y₁ and Y₂ are bridging groups, which can individually be, for example, —Cₙ—O—, wherein n is 0 or an integer of 1 or greater; and R₁ to R₃ independently hydrophilic polymeric portions, which can be the same or different, and have one or more pendent polymerizable groups.

In many aspects the branched compounds have one polymerizable group per polymeric branched portion (R) of the compound. In many aspects the polymerizable groups are located at the termini of the polymeric portions R.

A branched compound can be prepared from a polyol, such as a low molecular weight polyol (for example, a polyol having a molecular weight of 200 Da or less). In some aspects the branched compound can be derived from a triol, a tetraol, or other multifunctional alcohol. Exemplary polyol derivatives include derivatives of pentaerythritol, trimethylolpropane, and glycerol.

The polymeric portions of the branched compound can be selected from PVP, PEO, poly(ethyloxazoline), PPO, PAA and poly(meth)acrylic acid, PEG, and PEG-PPO, hydrophilic segmented urethanes, and polyvinyl alcohol, such as those described herein.

In some aspects, the branched component comprises one or more polymeric portions that is or are an oxyalkylene polymer, such as an ethylene glycol polymer.

For example, the preparation of a PEG-triacrylate macromer (trimethylolpropane ethoxylate (20/3 EO/OH) triacrylate macromer), which can be used as a component to form the biodegradable matrix, is described in Example 5 of commonly assigned U.S. Patent Application Publication No. 2004/0202774A1 (Chudzik, et al.).

The second macromer can be used in combination with the α(1→4)glucopyranose-based macromer at a final concentration sufficient to form a swellable degradable polymeric matrix. In many modes of practice the second macromer is present in the composition used to form the swellable biodegradable at a concentration in the range of about 5 mg/mL to about 350 mg/mL, about 10 mg/mL to about 300 mg/mL, about 20 mg/mL to about 250 mg/mL, or about 50 mg/mL to about 200 mg/mL. In some modes of practice, the α(1→4)glucopyranose-based macromer is used in an amount of about 150 mg/mL.

These concentration ranges represent the total amount of the second macromer is present in the composition (the composition also including the α(1→4)glucopyranose-based macromer). As discussed above; it should be recognized that in some modes of practice, the swellable biodegradable polymeric matrices are formed by redox polymerization, in which two independent liquid compositions are mixed, and upon mixing the macromers immediately polymerize to form the matrix. Given this, prior to mixing, the second macromer may be present in one of the compositions in an amount that is greater than the ranges described above (such as twice the concentration), but upon mixing the second macromer (prior to being incorporated into the matrix by polymerization) has a concentration within the ranges described above.

The amounts of macromer materials in the matrix-forming composition can also be described in terms of the weight ratio between the amounts of the α(1→4)glucopyranose-based macromer and second macromer (such as an alkylene oxide polymer-based macromer). In some modes of practice, the ratio (weight) between the α(1→4)glucopyranose-based macromer and second macromer is in the range of about 200:1 to about 1:10, about 50:1 to about 1:5, and more specifically in the range of about 10:1 to about 1:2. In one exemplary aspect the ratio is about 3:1.

The swellable biodegradable polymeric matrices can therefore have enzymatically degradable and non-degradable polymeric segments that are crosslinked via polymerized groups. Optionally, depending on the linkages between the polymeric portions and the polymerized groups, the polymeric matrix can also be non-enzymatically hydrolytically degradable, in addition to being enzymatically degradable.

The swellable biodegradable polymeric matrix can be placed at a target site in a dehydrated form, where it swells, or can be placed at the target site in a swollen form. The swollen polymeric matrix can then provide an effect at the target site, such as occlusion of the target area. Over a period of time, the swollen polymeric matrix degrades. The degradation can be caused entirely or partially by amylases causing the enzymatic degradation of the poly α(1→4)glucopyranose segments. Degradation of the poly α(1→4)glucopyranose segments also results in liberation of the polymeric segments formed from the second macromers, which can be eliminated from the body.

Depending on the linkage of the polymerizable groups (such as acrylate or methacrylate) groups, non-enzymatic hydrolysis of unsaturated esters may also occur, further promoting the degradation of the matrix.

Generally, the swellable degradable polymeric matrix is formed from a composition that includes, at least, the α(1→4)glucopyranose-based macromer and second macromer, and a polymerization initiator. Other, optional, components can be included in the composition to form the matrix. Some of these optional components are described herein.

Generally, the composition is prepared by dissolving or suspending the matrix-forming components in a suitable liquid. Suitable liquids include water, and other biocompatible polar liquids, such as alcohol. Combinations of polar liquids can also be used. The matrix-forming composition can have a viscosity that is suitable for the type of matrix-forming process performed.

Another way of describing the matrix-forming composition is by reference to the total amount matrix-forming materials in the composition. In many modes of practice the total amount of matrix forming material (including the α(1→4)glucopyranose-based macromer and the second macromer) is in the range of about 100 mg/mL to about 600 mg/mL, about 150 mg/mL to about 500 mg/mL, about 200 mg/mL to about 450 mg/mL, or about 250 mg/mL to about 400 mg/mL. In some modes of practice, the total amount of matrix forming material in the composition is about 350 mg/mL.

Typically, the composition includes an initiator that is capable of promoting the formation of a reactive species from a polymerizable group. For example, the initiator can promote a free radical reaction of the macromers present in the composition. In one embodiment the initiator is a compound that includes a photoreactive group (photoinitiator). For example, the photoreactive group can include an aryl ketone photogroup selected from acetophenone, benzophenone, anthraquinone, anthrone, anthrone-like heterocycles, and derivatives thereof.

In some aspects the photoinitiator includes one or more charged groups. The presence of charged groups can increase the solubility of the photoinitiator (which can contain photoreactive groups such as aryl ketones) in an aqueous system. Suitable charged groups include, for example, salts of organic acids, such as sulfonate, phosphonate, carboxylate, and the like, and onium groups, such as quaternary ammonium, sulfonium, phosphonium, protonated amine, and the like. Accordingly, a suitable photoinitiator can include, for example, one or more aryl ketone photogroups selected from acetophenone, benzophenone, anthraquinone, anthrone, anthrone-like heterocycles, and derivatives thereof; and one or more charged groups. Examples of these types of water-soluble photoinitiators have been described in U.S. Pat. No. 6,278,018.

Water-soluble polymerization initiators can be used at a concentration sufficient to initiate polymerization of the macromer components and formation of the matrix. For example, a water-soluble photo-initiator as described herein can be used at a concentration of about 0.5 mg/mL or greater. In some modes of practice, the photo-initiator is used at a concentration of about 1.0 mg/mL along with the matrix-forming components.

Thermally reactive initiators can also be used to promote the polymerization of hydrophilic polymers having pendent coupling groups. Examples of thermally reactive initiators include 4,4′ azobis(4-cyanopentanoic acid), 2,2-azobis[2-(2-imidazolin-2-yl)propane]dihydrochloride, and analogs of benzoyl peroxide. Redox initiators can also be used to promote the polymerization of the hydrophilic polymers having pendent coupling groups. In general, combinations of organic and inorganic oxidizers, and organic and inorganic reducing agents are used to generate radicals for polymerization. A description of redox initiation can be found in Principles of Polymerization, 2^{nd} Edition, Odian G., John Wiley and Sons, pgs 201-204, (1981).

Alternatively, formation of the swellable biodegradable polymeric matrix can be caused by the combination of an oxidizing agent/reducing agent pair, a “redox pair,” in the presence of the matrix-forming material.

Exemplary initiators include peroxides, including hydrogen peroxide, metal oxides, and oxidases, such as glucose oxidase. Exemplary activators include salts and derivatives of electropositive elemental metals such as Li, Na, Mg, Fe, Zn, Al, and reductases. Other reagents, such as metal or ammonium salts of persulfate, can be present in the composition to promote polymerization of the matrix-forming composition.

The redox pair can be combined in the presence of the matrix-forming material in any suitable manner. For example, a first composition containing the first component and the oxidizing agent, and a second composition including the reducing agent and the second component, can be prepared. Upon mixing of the first and second compositions polymerization commences and the swellable polymeric matrix begins to form.

In one mode of preparing the matrix, first and second compositions are held in separate chambers of dual syringe mixing device. When matrix formation is desired, simultaneous application of hand pressure to both syringe plungers in the device causes both the first and second compositions to flow from their respective syringes into a stationary mixing device (e.g., a “split flow” type mixer) where the compositions are mixed with one another at a predetermined ratio. After being mixed, the mixed composition exits the device though a single outlet orifice which can be positioned at the desired application site. Useful dual syringe mixing devices are commercially available under the trade designation MIXPAC™ from Mixpac Systems AG (Rotkreuz, CH).

The matrix-forming composition can also include one or more other ancillary reagent(s) that help promote formation of the matrix. These reagents can include polymerization co-initiators, reducing agents, and/or polymerization accelerators known in the art. These ancillary agents can be included in the composition at any useful concentration.

Exemplary co-initiators include organic peroxides, such as those that are derivatives of hydrogen peroxides (H₂O₂) in which one or both of the hydrogen atoms are replaced by an organic group. Organic peroxides contain the —O—O— bond within the molecular structure, and the chemical properties of the peroxides originate from this bond. The peroxide polymerization co-initiator can be a stable organic peroxide, such as an alkyl hydroperoxide. Exemplary alkyl hydroperoxides include t-butyl hydroperoxide, p-diisopropylbenzene peroxide, cumene hydroperoxide, acetyl peroxide, t-amyl hydrogen peroxide, and cumyl hydrogen peroxide.

Other polymerization co-initiators include azo compounds such as 2-azobis(isobutyronitrile), ammonium persulfate, and potassium persulfate.

The matrix-forming composition can include a reducing agent such as a tertiary amine. In many cases the reducing agent, such as a tertiary amine, can improve free radical generation. Examples of the amine compound include primary amines such as n-butylamine; secondary amines such as diphenylamine; aliphatic tertiary amines such as triethylamine; and aromatic tertiary amines such as p-dimethylaminobenzoic acid.

In other aspects of the invention, in addition to these components, the composition used to form the swellable polymeric matrix can include one or more polymerization accelerator(s). A polymerization accelerator such as n-vinyl pyrrolidone can be used. In some aspects a polymerization accelerator having a biocompatible functional group (e.g., a biocompatible polymerization accelerator) is included in the composition of the present invention. The biocompatible polymerization accelerator can also include an N-vinyl group such as N-vinyl amide group. Biocompatible polymerization accelerators are described in commonly assigned U.S. Patent Application Publication No. 2005/0112086.

Generally, matrix formation takes place by activating the polymerization initiator, which causes the free-radical polymerization of the macromer components in the composition. Light sources, including UV and short wave visible light sources can be used to activate photoinitiators, and the appropriate source can be chosen based on the activation wavelength of the photoinitiator used. Activation of the polymerization composition can be performed away from the body, or at a target site in the body (i.e., in situ polymerization).

In some aspects, a “pre-formed” swellable polymeric matrix is prepared, referring to those matrices that are not formed in situ, but rather away from a tissue site. A pre-formed swellable polymeric matrix can have a defined structure. A pre-formed matrix can be created using a mold or casting so the matrix can be made into a particular shape. Alternatively, a pre-formed matrix can be created and then shaped as desired, by a process such as cutting. Exemplary shapes useful for tissue treatment include, but are not limited to, spherical, cylindrical, clam-shell, flattened, rectangular, square, and rounded shapes.

In some aspects of the invention, the swellable polymeric matrix is formed in association with a medical device. For example, the matrix can be formed as an overmold or a coating in association with a part of, or the entire device.

A “coating” refers to one or more layers of matrix material, formed by applying the matrix forming materials to all or a portion of a surface of an article by conventional coating techniques.

An “overmold” refers to matrix material formed in association with all or a portion of a surface of an article. An overmold of matrix material is generally thicker than a coating, and typically formed using a molding process rather than a coating process.

A “medical device” refers to an article used in a medical procedure. Typically, the matrix is formed on the surface of an implantable medical device. From a structural standpoint, the implantable medical device may be a simple article, such as a rod, pellet, sphere, or wire, on which the swellable biodegradable matrix can be formed. The implantable medical device can also have a more complex structure or geometry, as would be found in an intralumenal prosthesis, such as a stent.

An implantable device having a swellable biodegradable polymeric matrix (formed using a combination the α(1→4)glucopyranose-based macromer and the second macromer), or a portion thereof, can be configured to be placed within the vasculature (an implantable vascular device), such as in an artery, vein, fistula, or aneurysm. In some cases the implantable device is an occlusion device selected from vascular occlusion coils, wires, or strings that can be inserted into aneurysms. Some specific vascular occlusion devices include detachable embolization coils. In some cases the implantable device is a stent.

Other medical articles on which the swellable biodegradable polymeric matrix can be formed include, but are not limited to, small diameter grafts, abdominal aortic aneurysm grafts; wound dressings and wound management devices; hemostatic barriers; mesh and hernia plugs; patches, including uterine bleeding patches, atrial septic defect (ASD) patches, patent foramen ovale (PFO) patches, ventricular septal defect (VSD) patches, and other generic cardiac patches; ASD, PFO, and VSD closures; percutaneous closure devices; birth control devices; breast implants; orthopedic devices such as orthopedic joint implants, bone repair/augmentation devices, cartilage repair devices; urological devices and urethral devices such as urological implants, and bladder devices.

Implantable medical devices can be prepared from metals such as platinum, gold, or tungsten, although other metals such as rhenium, palladium, rhodium, ruthenium, titanium, nickel, and alloys of these metals, such as stainless steel, titanium/nickel, and nitinol alloys, can be used.

The surface of metal-containing medical devices can be pretreated (for example, with a Parylene™-containing coating composition) in order to alter the surface properties of the biomaterial, when desired. Metal surfaces can also be treated with silane reagents, such as hydroxy- or chloro-silanes.

Implantable medical devices can also be partially or entirely fabricated from a plastic polymer. In this regard, the swellable polymeric matrix can be formed on a plastic surface. Plastic polymers include those formed of synthetic polymers, including oligomers, homopolymers, and copolymers resulting from either addition or condensation polymerizations. Examples of suitable addition polymers include, but are not limited to, acrylics such as those polymerized from methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide; vinyls such as ethylene, propylene, vinyl chloride, vinyl acetate, vinyl pyrrolidone, vinylidene difluoride, and styrene. Examples of condensation polymers include, but are not limited to, nylons such as polycaprolactam, polylauryl lactam, polyhexamethylene adipamide, and polyhexamethylene dodecanediamide, and also polyurethanes, polycarbonates, polyamides, polysulfones, poly(ethylene terephthalate), polydimethylsiloxanes, and polyetherketone.

A medical device with a swellable biodegradable polymeric matrix “overmold” can be formed in a process using a mold, a composition comprising the matrix-forming material, and a medical device. The medical device can be placed in a portion of the mold so the composition can be placed in contact with all or a portion of the surface of the device. For example, a device in the shape of a rod or coil is fixtured in a mold so that that composition can be in contact with the entire surface of the device. The composition can then be added to mold and treated to promote matrix formation. In some cases, the mold is made of a material that allows UV light to pass through it, and the composition can include a photo-initiator, which is activated by the UV and causes matrix formation.

In another exemplary mode of preparation, a matrix overmold can be formed by adding the composition to the mold and then partially polymerizing the matrix so the composition increases in viscosity. The medical device can then be placed in the partially polymerized composition, and due to its increased viscosity, suspends the device within the composition as desired. The composition can then be fully polymerized to solidify the materials of the composition, which forms the swellable biodegradable polymeric matrix as an overmold on the device. After the swellable biodegradable polymeric matrix forms as an overmold, the device can be removed from the mold.

The weight of the polymeric matrix can be a substantial percentage of the weight of the overall device. When the matrix is in a partially hydrated for fully hydrated state, it can have a weight that is substantially greater than the device which it overmolds.

The overmold can be formed on any desired medical device and can have dimensions suitable for occluding a target site in the body. The swellable biodegradable polymeric matrix in an overmold is typically thicker than the matrix of a coating, which can provide advantages for occlusion of a target site.

In some aspects, the matrix has a thickness in the range of about 50 μm to about 500 μm, and more specifically in the range of about 100 μm to about 300 μm. The matrix can then be dried to have a thickness in the range of about 25 μm to about 400 μm, respectively, and more specifically in the range of about 75 μm to about 250 μm, respectively. The matrix can be hydrated (e.g., in vivo), which can swell the matrix to a thickness in the range of about 100 μm to about 2500 μm, respectively, and more specifically in the range of about 750 μm to about 1500 μm, respectively.

As a specific example, in the case of an implantable device having a diameter of about 0.5 mm, a swellable polymeric matrix in the form of an overmold is formed on the device. The overmold has a thickness in the range of about 100 μm to about 450 μm, or about 100 μm to about 300 μm in a dried state. During and/or after delivery of the article to the target site, the coating swells to have a thickness in the range of about 750 μm to about 1500 μm, causing occlusion of the target site.

A device with an overmolding can be delivered to a target site in the body, where it hydrates to a hydrogel within the target site. Delivery of the device can be performed using a catheter and/or other guide instruments, such as guidewires.

The swellable polymeric matrix can become hydrated in a relatively short period of time, such as period of time in the range of about 30 minutes to about 2 hours, or about 1 hour. Swelling of the polymeric matrix can be monitored to determine if the hydrogel occludes the target site as desired.

In another aspect, the swellable polymeric matrix is in the form of a coating on a medical device. A matrix coating that includes the first and second macromers can be formed various ways.

In one mode of practice, a composition including the first and second macromers is dip-coated onto the surface of the substrate to form a coating. The composition on the surface can then be treated to cause matrix formation. For example, a composition including the first and second macromers, and a photoactivatable polymerization initiator is dip-coated on the surface of a device. During and/or after the dip-coating step, the applied material can be irradiated to promote polymerization of the first and second components, and matrix formation.

Other techniques, such as brushing or spraying the composition can be used to form the coating. The method of spray coating can be performed by spraying the composition on the surface the device, and then treating the composition to form the coating.

In another aspect of the invention, the α(1→4)glucopyranose-based macromer and the second macromer are used to form an implantable article, without requiring the presence of a non-hydrogel material to be associated with the matrix. In other words, the swellable matrix itself forms the implantable article.

Such an implantable matrix article can have a simple or a complex geometry. A simple geometry is exemplified by a device that is in the form of a filament (e.g., threads, strings, rods, etc.). A matrix article with a simple geometry can be prepared by various methods. One method for forming the matrix article uses the same process as used to form the overmolded device, but does not include a device within the mold. Again, the mold can be, for example, a piece of tubing which has an inner area corresponding to the first configuration of the body member. The composition can then be injected into the tubing to fill the tubing. The composition in the tubing can then be treated to activate the polymerization initiator (such as by photo-initiated polymerization). Polymerization promotes crosslinking of the α(1→4)glucopyranose-based macromer and the second macromer (and any other optional polymerizable material) and establishes a polymeric matrix in the configuration of the mold.

In many cases, the matrix article can be used in the same way that the overmolded device is used.

The polymerizable materials of the present invention can also be used for the formation of an in situ polymerized mass at a target site in the body. Generally, a composition that includes the α(1→4)glucopyranose-based macromer and the second macromer can be delivered to or applied to a target site, and then the composition is treated to promote polymerization and formation of the swellable biodegradable polymeric matrix. In some cases, two separate solutions (for example, each having a member of a redox pair) are delivered to, and mixed at a target site in situ. The mixing of the solutions causes polymerization and formation of the swellable biodegradable polymeric matrix at the target site. In some methods of treatment, the matrix formed at the target site hydrates to occlude the target site.

In some modes of practice, a composition containing the polymerizable materials can be passed through a small gauge delivery conduit to place the composition at a target site. Polymerization and matrix formation can occur in situ. Delivering a polymerizable composition to the target site (such as a neuroaneurysm) can be performed using a microcatheter. Microcatheters generally have very small diameters, such as about 5 french (fr) or less. (“French size” generally refers to units of outer diameter of a catheter; Fr size×0.33=outer diameter of the catheter in mm.) Exemplary microcatheters having a size of about 2.3 french or less, such as in the range of about 1.7 french to about 2.3 french are commercially available from, for example, Boston Scientific (e.g., Excelsior™ SL-10).

As an example, the biodegradable matrix is formed in situ by either preparing a matrix-forming composition and then delivering it to the site via a microcatheter, or independently delivering two compositions to the target site where they mix, causing polymerization of the macromers and matrix formation. For example, compositions can be mixed prior to delivering through a microcatheter. In many aspects polymerization of the macromer-containing composition is initiated using redox polymerization initiators, such as described herein. Prior to be introduced into a single lumen catheter, a mixed composition is prepared using a mixing device, such as the MIXPAC™ device (Mixpac Systems AG), discussed herein.

If two compositions are separately delivered to a target site, a dual lumen microcatheter can be used.

The swellable polymeric matrices of the present invention can also include a bioactive agent in polymeric matrix. In some preparations, the bioactive agent can be released during the degradation of the matrix. Examples of bioactive agents that can be included in the matrix include, but are not limited to: ACE inhibitors, actin inhibitors, analgesics, anesthetics, anti-hypertensives, anti polymerases, antisecretory agents, anti-AIDS substances, antibiotics, anti-cancer substances, anti-cholinergics, anti-coagulants, anti-convulsants, anti-depressants, anti-emetics, antifungals, anti-glaucoma solutes, antihistamines, antihypertensive agents, anti-inflammatory agents (such as NSAIDs), anti metabolites, antimitotics, antioxidizing agents, anti-parasite and/or anti-Parkinson substances, antiproliferatives (including antiangiogenesis agents), anti-protozoal solutes, anti-psychotic substances, anti-pyretics, antiseptics, anti-spasmodics, antiviral agents, calcium channel blockers, cell response modifiers, chelators, chemotherapeutic agents, dopamine agonists, extracellular matrix components, fibrinolytic agents, free radical scavengers, growth hormone antagonists, hypnotics, immunosuppressive agents, immunotoxins, inhibitors of surface glycoprotein receptors, microtubule inhibitors, miotics, muscle contractants, muscle relaxants, neurotoxins, neurotransmitters, polynucleotides and derivatives thereof, opioids, photodynamic therapy agents, prostaglandins, remodeling inhibitors, statins, steroids, thrombolytic agents, tranquilizers, vasodilators, growth factors, and vasospasm inhibitors. One or more bioactive agents can be present in the polymeric matrix in an amount sufficient to provide a desired biological response.

In some aspects of the invention, the matrix includes a bioactive agent that is a macromolecule. Exemplary macromolecules can be selected from the group consisting of polynucleotides, polysaccharides, and polypeptides. In some aspects the bioactive agent has a molecular weight of about 1000 Da or greater.

One class of bioactive agents that can be released from the matrix includes polynucleotides. As used herein “polynucleotides” includes polymers of two or more monomeric nucleotides. Nucleotides can be selected from naturally occurring nucleotides as found in DNA (adenine, thymine, guanine, and cytosine-based deoxyribonucleotides) and RNA (adenine, uracil, guanine, and cytosine-based ribonucleotides), as well as non-natural or synthetic nucleotides.

Types of polynucleotides that can be released from the matrix include plasmids, phages, cosmids, episomes, integratable DNA fragments, antisense oligonucleotides, antisense DNA and RNA, aptamers, modified DNA and RNA, iRNA (immune ribonucleic acid), ribozymes, siRNA (small interfering RNA), miRNA (micro RNA), locked nucleic acids and shRNA (short hairpin RNA).

If it is desired to include a bioactive agent in the matrix, one of various methods can be performed to provide form a bioactive agent-containing matrix. For example, in some modes of practice, the bioactive agent is dissolved in a matrix-forming composition and then the macromers of the composition are polymerized, entrapping the bioactive agent in the matrix. Following placement at a target site in the body, the bioactive agent can be released by degradation of the matrix and/or diffusion of the bioactive agent out of the matrix.

In another mode of practice, the bioactive agent is suspended in the matrix forming composition, and the macromers of the composition are polymerized, which also results of the bioactive agent being entrapped in the matrix.

In some cases, the bioactive agent can be present in the matrix in particulate form. “Particulate form,” generally refers to small particles of bioactive agent. Small particles of bioactive agent can be formed by processes such as micronizing, milling, grinding, crushing, and chopping a solid mass of bioactive agent. Particulates of bioactive agent can be from a powdered composition of the bioactive agent. In some cases, powders of bioactive agent can be formed from processes including precipitation and/or crystallization, and spray drying. Particulates of bioactive agent can be in the form of microparticles or microspheres. The microparticles of bioactive agent can comprise any three-dimensional structure that can be immobilized in the matrix formed by the macromers described herein. Microspheres are microparticles that are spherical or substantially spherical in shape.

Bioactive agent-containing microparticles can be formed substantially or entirely of bioactive agent, or the microparticles can include a combination of a bioactive agent and a non-active agent, such as an excipient compound or polymeric material.

Microparticles formed solely of one or more bioactive agents have been described in the art. For example, the preparation of paclitaxel microparticles has been described in U.S. Pat. No. 6,610,317. Therefore the bioactive agent can be a low molecular weight compound present. As another example, the microparticle is formed from a macromolecular compound, such as a polypeptide. Polypeptide microparticles are described in commonly-assigned copending U.S. patent application Ser. No. 12/215,504 filed Jun. 27, 2008, (Slager, et al.).

In some aspects, the swellable biodegradable polymeric matrix can also include a pro-fibrotic agent. A pro-fibrotic agent can promote a rapid and localized fibrotic response in the vicinity of the hydrogel. This can lead to the accumulation of clotting factors and formation of a fibrin clot in association with the hydrogel. In some aspects the pro-fibrotic agent is a polymer. The polymer can be based on a natural polymer, such as collagen, or a synthetic polymer.

Optionally, the bioactive agent can be coupled to a polymeric segment which forms the matrix. For example, a bioactive agent can include polymerizable groups and can be reacted along with the α(1→4)glucopyranose-based macromer and the second macromer to be covalently incorporated into the matrix. An example of a matrix protein-based macromer is a collagen macromer, which is described in Example 12 of commonly assigned U.S. Pub. No. US-2006/0105012A1.

Alternatively, a bioactive agent can be covalently coupled to a polymeric segment via a reacted photogroup. For example, photogroup-derivatized matrix proteins, such as photo-collagen (described in commonly-assigned U.S. Pat. No. 5,744,515) and activated to bond collagen to the polymeric material forming the matrix.

The swellable biodegradable polymeric matrix can also include an imaging material. Imaging materials can facilitate visualization of the polymeric matrix one implanted or formed in the body. Medical imaging materials are well known. Exemplary imaging materials include paramagnetic material, such as nanoparticular iron oxide, Gd, or Mn, a radioisotope, and non-toxic radio-opaque markers (for example, cage barium sulfate and bismuth trioxide). Radiopacifiers (such as radio opaque materials) can be included in a composition used to make the matrix. The degree of radiopacity contrast can be altered by controlling the concentration of the radiopacifier within the matrix. Common radio opaque materials include barium sulfate, bismuth subcarbonate, and zirconium dioxide. Other radio opaque materials include cadmium, tungsten, gold, tantalum, bismuth, platinum, iridium, and rhodium.

Paramagnetic resonance imaging, ultrasonic imaging, x-ray means, fluoroscopy, or other suitable detection techniques can detect the swellable or swollen matrices that include these materials. As another example, microparticles that contain a vapor phase chemical can be included in the matrix and used for ultrasonic imaging. Useful vapor phase chemicals include perfluorohydrocarbons, such as perfluoropentane and perfluorohexane, which are described in U.S. Pat. No. 5,558,854; other vapor phase chemicals useful for ultrasonic imaging can be found in U.S. Pat. No. 6,261,537.

Testing can be carried out to determine mechanical properties of the matrix. Dynamic mechanical thermal testing can provide information on the viscoelastic and rheological properties of the matrix by measuring its mechanical response as it is deformed under stress. Measurements can include determinations of compressive modulus, and shear modulus. Key viscoeslatic parameters (including compressive modulus and sheer modulus) can be measured in oscillation as a function of stress, strain, frequency, temperature, or time. Commercially available rheometers (for example, available from (TA Instruments, New Castle, Del.) can be used to make these measurements. The testing of hydrogels for mechanical properties is also described in Anseth et al. (1996) Mechanical properties of hydrogels and their experimental determination, Biomaterials, 17:1647.

The matrix can be measured to determine its complex dynamic modulus (G*): G*=G′+iG″=σ*/γ*, where G′ is the real (elastic or storage) modulus, and G″ is the imaginary (viscous or loss) modulus, these definitions are applicable to testing in the shear mode, where G refers to the shear modulus, σ to the shear stress, and γ to the shear strain.

The hydrogel can also be measured for its swelling (or osmotic) pressure. Commercially available texture analyzers (for example, available from Stable Micro Systems; distributed by Texture Technologies Corp; Scarsdale, N.Y.) can be used to make these measurements. Texture analyzers can allow measurement of force and distance in tension or compression.

In some modes of practice, the hydrogels having swelling pressures of about 10 kPa (about 100 g/cm²) or greater, such as in the range of about 10 kPa to about 750 kPa (about 7600 g/cm²), or about 10 kPa to 196 kPa (2000 g/cm²) are used. In other words, the matrix is capable of exerting a swelling force in these ranges upon hydration from a dehydrated or partially hydrated form.

In some formations, the polymeric matrix is capable of swelling in water to a weight in the range of about 1.5 or greater its weight in a dehydrated form, such as in the range of about 1.5 to about 10 times its weight in a dehydrated form.

In some formations, the polymeric matrix is capable of swelling in water to a size that is at least 25% greater than its size in a dehydrated form, such as in the range of about 25% greater to about 150% greater than its size in a dehydrated form, or about 45% greater to about 80% greater than its size in a dehydrated form.

### Examples 1-12

### Swellable Degradable Matrices Formed from Maltodextrin Methacrylate and Various Biocompatible Biostable Macromers

A solution of 1 mg/mL photoinitiator 4,5-bis(4-benzoylphenylmethyleneoxy)benzene-1,3-disulfonic acid (5 mg) (DBDS) (as described in U.S. Pat. No. 6,278,018 (Example 1) and commercially available from SurModics, Inc. (Eden Prairie, Minn.)) was prepared. Maltodextrin methacrylate (MD-MA) with a methacrylate load of ˜0.18 (0.18 mmol/gram, methacrylate/maltodextrin) was dissolved at a concentration of 350 mg/mL into the DBDS solution. The PPG or PEG-based biocompatible biostable macromers (as described in Examples 1-10, Table 1) were then dissolved into the DBDS solution at concentrations of 350 mg/mL. Photo-PA-PEG-AMPS and photo-PA (described below) were dissolved in the DBDS solution at 200 mg/mL and 100 mg/mL, respectively.

Matrix-forming solutions were prepared by mixing 400 μL of the MD-MA/DBDS solutions with 100 μL of the biocompatible polymer/DBDS solutions, individually. The final concentrations of the macromers/polymers are reflected in Table 1. The matrix-forming solutions were then vortexed for 30 seconds. Approximately 50 μL of each matrix-forming solution was pipetted into silicone tubing with a 1.58 mm ID. The remaining matrix-forming solution was pipetted into a well on a 24-well plate. Both the plate and tubing were placed under UV light for 120 seconds to cure.

The filaments were removed from the silicone tubing and were allowed to dry in a humidity-controlled room for 48 hours, upon which diameter measurements were made The diameter of the polymer filament was measured using a Leica MZ12₅ stereomicroscope with Techniquip™ lighting and ImagePro™-Plus software version 6.1. The filaments were allowed to swell for 2 hours in sterile water at 23 C to obtain the respective swelling data.

The physical properties of the gels were determined by compression force testing and swellability testing. Compressive force of the gels was tested using a TAXT2™ texture analyzer with 5 mm diameter ball probe was used to determined compression strength. The procedure used a test speed of 0.5 mm/sec and a trigger force of 4 g. The probe compressed to 25% of the depth of the material as compared to the calibration depth.

Poly(propylene glycol)₅₄₀-diacrylate (PPG₅₄₀-DA), poly(propylene glycol)₉₀₀-diacrylate (PPG₉₀₀-DA), poly(ethylene glycol)₂₅₀-diacrylate (PEG₂₅₀-DA), poly(ethylene glycol)₅₇₅-diacrylate (PEG₅₇₅-DA), poly(ethylene glycol)₅₅₀-dimethacrylate (PEG₅₅₀-DMA), poly(ethylene glycol)₁₀₀₀-dimethacrylate (PEG₁₀₀₀-DMA), poly(ethylene glycol)₇₅₀-dimethacrylate (PEG₇₅₀-DMA), poly(ethylene glycol)₇₀₀-diacrylate (PEG₇₀₀-DA), poly(ethylene glycol)₄₅₀-monoetheracrylate (PEG₄₅₀-MEA) were obtained from Sigma-Aldrich (St. Louis, Mo.) or Polysciences (Warrington, Pa.).

The photogroup-derivatized polymer photo-PA-PEG-AMPS (SurModics, Inc., Eden Prairie, Minn.) was prepared by a copolymerization of acrylamide, methoxy poly(ethylene glycol), 2-acrylamide-2-methylpropanesulfonic acid (AMPS), and N-(3-aminopropyl)methacrylamide (APMA) using water as the solvent. Purification after this step was performed using dialysis. Photo-loading was performed secondly using BBA (4-benzoylbenzoyl chloride) in a mixed aqueous/organic solvent under Schotten-Baumann conditions. Residual amines left after the photoderivatization were capped using acetic anhydride. Final purification was done using dialysis and dried by lyophilization. The resulting monomer is Acetylated PA-(10% w/w)methoxy-PEG1000-MMA-(5%)AMPS-(1%)APMA-(0.015)BBA.

The photogroup-derivatized polymer photo-polyacrylamide (photo-PA) was prepared as follows. A photo-derivatized acrylamide monomer (BBA-APMA) was prepared by the reaction of N-(3-aminopropyl)methacrylamide (APMA) and benzoylbenzoyl chloride (BBA-Cl) followed by purification by recrystallization. The BBA-APMA monomer was then copolymerized with acrylamide in tetrahydrofuran (THF) resulting in formation of a white precipitate. The solid was filtered, dialyzed and lyophilized to give the final product. The resulting polymer was polyacrylamide-(3.5%)BBA-APMA-(0.4)BBA (photo-PA).

TABLE 1

| | First | Second | Force | |
|---|---|---|---|---|
| Example | Polymer | Polymer | (g) | Swelling |
| 1 | MD-MA | MD-MA | 111.85 | 43.4% |
| | (280 mg/mL) | (70 mg/mL) | | |
| 2 | MD-MA | PEG₇₀₀-DA | 115.62 | 66.9% |
| | (280 mg/mL) | (70 mg/mL) | | |
| 3 | MD-MA | PPG₉₀₀-DA | 83.46 | 53.0% |
| | (280 mg/mL) | (70 mg/mL) | | |
| 4 | MD-MA | PEG₇₅₀-DMA | 70.56 | 63.1% |
| | (280 mg/mL) | (70 mg/mL) | | |
| 5 | MD-MA | PEG₂₅₀-DA | 91.83 | 67.4% |
| | (280 mg/mL) | (70 mg/mL) | | |
| 6 | MD-MA | PPG₅₄₀-DA | 69.88 | 45.2% |
| | (280 mg/mL) | (70 mg/mL) | | |
| 7 | MD-MA | PEG₁₀₀₀-DMA | 84.97 | 58.2% |
| | (280 mg/mL) | (70 mg/mL) | | |
| 8 | MD-MA | PEG₅₅₀-DMA | 78.11 | 60.3% |
| | (280 mg/mL) | (70 mg/mL) | | |
| 9 | MD-MA | PEG₄₅₀-MEA | 86.42 | 75.8% |
| | (280 mg/mL) | (70 mg/mL) | | |
| 10 | MD-MA | PEG₅₇₅-DA | 90.32 | 40.6% |
| | (280 mg/mL) | (70 mg/mL) | | |
| 11 | MD-MA | photo-PA-PEG-AMPS | 59.92 | 58.7% |
| | (280 mg/mL) | (40 mg/mL) | | |
| 12 | MD-MA | Photo-PA | 61.49 | 58.3% |
| | (280 mg/mL) | (20 mg/mL) | | |

### Example 13

### Degradable Swellable Matrix with Redox Initiation for In Situ Formation

A 6 mM hydrogen peroxide solution and 6 mM ferrous lactate solution were freshly prepared into separate sterile conical tubes. Into a two glass vials, 350 mg of maltodextrin methacrylate (MD-MA) was weighed into each. Next, 70 mg of poly(ethylene glycol)₇₀₀ diacrylate was added to each of the vials containing the MD-MA. To the first vial, 1 mL of the hydrogen peroxide solution was added. To the second, 1 mL of the ferrous lactate solution was added. Both vials were vortexed for 30 seconds to fully dissolve the reagents. Each solution was than evacuated into a 3 mL syringe with and air bubbles were removed. A Micromedics FibriJet™ blending connector was added to the two syringes. A 21 gauge needle was attached to the end of the blending connector and the Micromedics housing kit was attached to ensure even distribution of the materials when combined. The solutions were ejected simultaneously into 1.99 mm inner diameter silicone tubing, mimicking an application into a vessel or tube in the body. The macromer components polymerized to a hydrogel in less than 5 seconds.

### Examples 14-16

### Amylase-Mediated Degradation of MD-MA/PEG₇₀₀-DA Matrices

Amylase-mediated degradation studies were performed on matrices prepared from maltodextrin methacrylate and poly(ethylene glycol)₇₀₀-diacrylate (PEG₇₀₀-DA). A DBDS solution was prepared as described in Examples 1-12, and MD-MA was dissolved in a 1 mg/mL DBDS solution to a concentration of 350 mg/mL. Separately, poly(ethylene glycol)₇₀₀-diacrylate (PEG₇₀₀-DA) was dissolved in a 1 mg/mL DBDS solution to a concentration of 350 mg/mL.

Matrix-forming solutions were prepared by mixing various amounts of the MD-MA/DBDS solution with the PEG₇₀₀-DA/DBDS solution to provide matrix-forming solutions with the final concentrations of MD-MA and PEG₇₀₀-DA as described in Table 2 (Example 14: 90% MD/10% PEG; Example 15: 70% MD/30% PEG and Example 16: 50% MD/50% PEG). All formulations were vortexed before use.

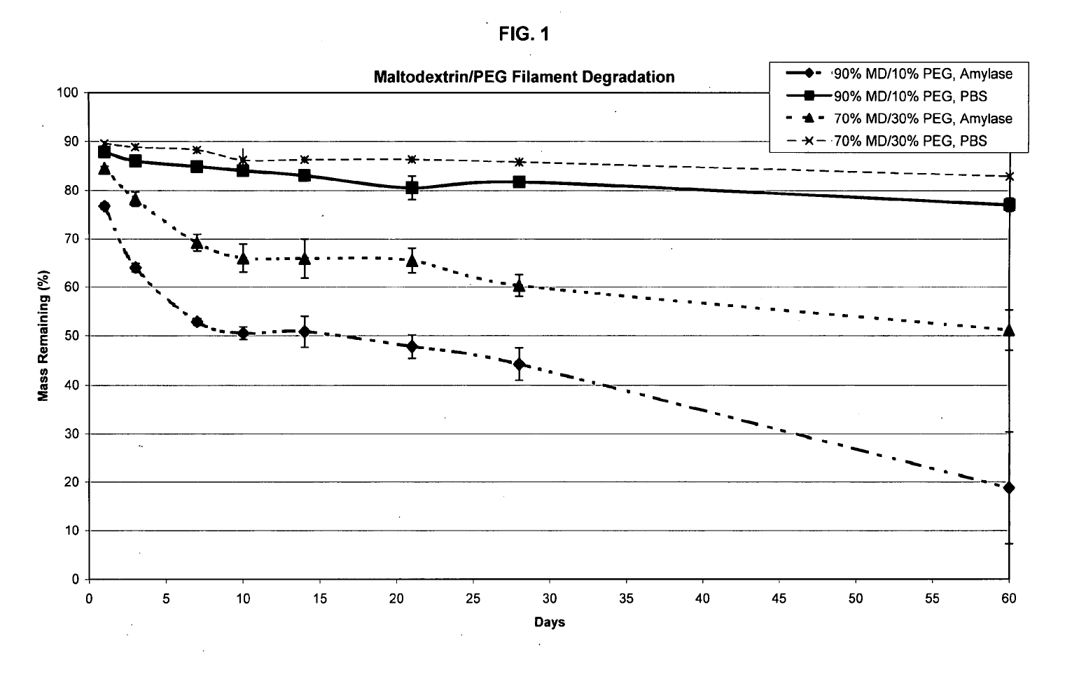

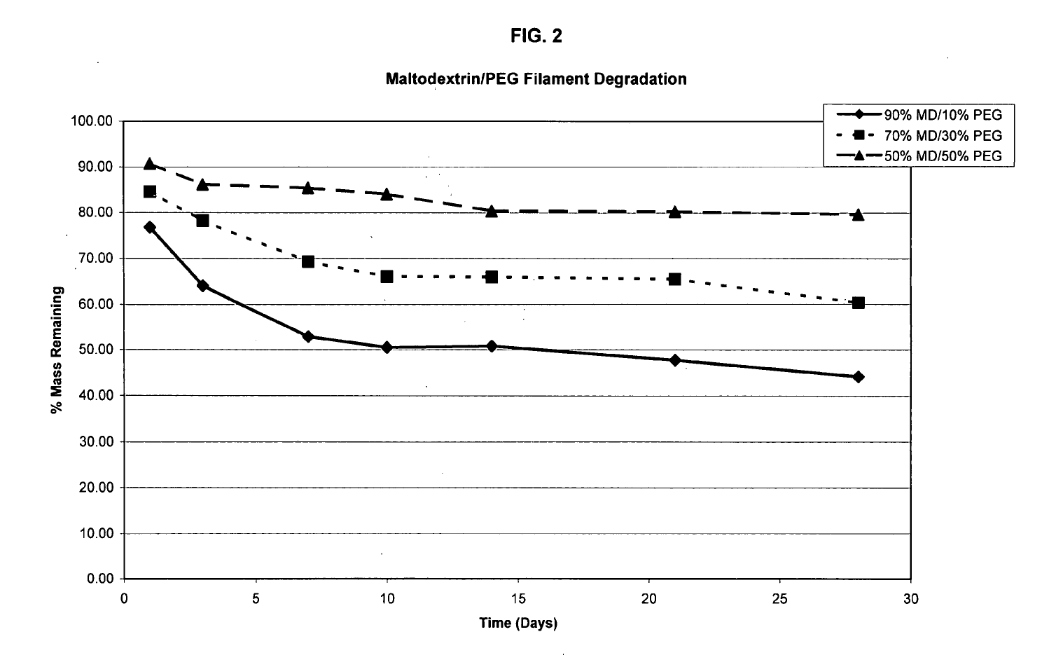

Into 1.47 mm I.D. silicone tubing (HelixMark), each formulation was injected, filling the tubes with liquid. The silicone tubing with solution was then placed under UV light for 2 minutes to cure. When complete, each polymerized hydrogel was removed from the tubing and cut into 1 cm lengths. The filaments were dried completely over 3 days in a humidity-controlled atmosphere and initial weight measurements were taken. The filaments were then placed into individual vials, which were filled with a control solution (1 mM PBS with 30 mM calcium chloride) or an amylase solution (16×amylase in 1 mM PBS with 30 mM calcium chloride). The degradation study was conducted at a temperature of 37° C. and carried out over a time period of 28 days. The amylase solution was made fresh every week and changed twice per week. At predetermined time points, the filaments were removed from the control and amylase solutions and weighed. The percentage mass remaining of the filaments at these time points was then calculated. FIG. 1 shows degradation of the 90% MD/10% PEG and 70% MD/30% PEG filaments in the control and amylase solutions over the course of the degradation study. FIG. 2 shows degradation of the 90% MD/10% PEG, 70% MD/30% PEG, and 50% MD/50% PEG filaments in the amylase solutions over the course of the degradation study.

TABLE 2

| Example | First Polymer | Second Polymer |
|---|---|---|
| 14 | MD-MA (315 mg/mL) | PEG₇₀₀-DA (35 mg/mL) |
| 15 | MD-MA (245 mg/mL) | PEG₇₀₀-DA (105 mg/mL) |
| 16 | MD-MA (175 mg/mL) | PEG₇₀₀-DA (175 mg/mL) |

1. A biocompatible biodegradable swellable or swollen polymeric matrix comprising first and second polymer-containing segments crosslinked via polymerized groups, wherein the first polymer-containing segment comprises poly-α(1→4)glucopyranose
and the second polymer-containing segment comprises a biocompatible biostable hydrophilic polymer.

2. The biocompatible biodegradable swellable or swollen polymeric matrix of claim 1 wherein polymerized groups are pendent from the poly-α(1→4)glucopyranose in an amount in the range of 0.05 mmol/gram to 0.7 mmol/gram (polymerized groups/poly-α(1→4)glucopyranose).

3. (canceled)

4. The biocompatible biodegradable swellable or swollen polymeric matrix of claim 1 wherein the first polymer-containing segment comprises poly-α(1→4)glucopyranose having a molecular weight of 500,000 Da or less.

5. The biocompatible biodegradable swellable or swollen polymeric matrix of claim 1 wherein the second polymer-containing segment comprises an oxyalkylene polymer.

6. (canceled)

7. The biocompatible biodegradable swellable or swollen polymeric matrix of claim 1 wherein the second polymer-containing segment comprises a polymer selected from the group consisting of poly(ethylene oxide) (PEO), poly(ethyloxazoline), poly(propylene oxide) (PPO), poly(ethylene glycol) (PEG), and copolymers of polyethylene glycol and polypropylene oxide (PEG-PPO).

8. (canceled)

9. The biocompatible biodegradable swellable or swollen polymeric matrix of claim 1 wherein the second polymer-containing segment has termini, and the polymerized groups are present at the termini.

10. The biocompatible biodegradable swellable or swollen polymeric matrix of claim 1 wherein the second polymer-containing segment has a molecular weight in the range of 100 Da to 40 kDa.

11. (canceled)

12. The biocompatible biodegradable swellable or swollen polymeric matrix of claim 1 wherein the first polymer-containing segment and the second polymer-containing segment are present in the matrix at a weight ratio in the range of 200:1 to 1:10, respectively.

13. (canceled)

14. The biocompatible biodegradable swellable polymeric matrix of claim 1 which is capable of swelling in water to a weight of 1.5 times or greater a weight of the matrix in a dehydrated form.

15. The biocompatible biodegradable swellable polymeric matrix of claim 1 which exerts a swelling force of 100 g/cm² or greater upon hydration from a dehydrated form.

16. The biocompatible biodegradable swellable polymeric matrix of claim 1 which is capable of swelling in water to a size that is at least 25% greater than its size in a dehydrated form.

17. The biocompatible biodegradable swellable or swollen polymeric matrix of claim 1 which is associated with an implantable medical device.

18. The biocompatible biodegradable swellable or swollen polymeric matrix of claim 1 comprising a bioactive agent.

19. (canceled)

20. (canceled)

21. A method for forming a biocompatible biodegradable swellable or swollen polymeric matrix comprising steps of:
(a) providing a composition comprising
(i) poly-α(1→4)glucopyranose comprising pendent polymerizable groups, and
(ii) a biocompatible biostable hydrophilic polymer comprising a pendent polymerizable group, and
(b) activating the polymerizable groups to cause crosslinking of the poly-α(1→4)glucopyranose and biocompatible biostable hydrophilic polymer and matrix formation.

22. The method of claim 21 wherein the composition comprises poly-α(1→4)glucopyranose in an amount in the range of 50 mg/mL to about 600 mg/mL.

23. (canceled)

24. The method of claim 21 wherein the composition comprises the biocompatible biostable hydrophilic polymer in an amount in the range of 5 mg/mL to about 350 mg/mL.

25. (canceled)

26. The method of claim 21 where, in step (a) the composition is provided to a target location on a subject, and in step (b) the polymerizable groups are activated to cause crosslinking and in situ matrix formation.

27. A method for treating a subject comprising a step of placing a biocompatible biodegradable swellable or swollen polymeric matrix according to claim 1 at a target location in a subject.

28. The method of claim 27 resulting in a swollen polymeric matrix which occludes the target location.

29. The method of claim 26 wherein the polymeric matrix swells or is swollen at the target site, and the matrix is allowed to contact amylase for a period of time and the amylase promotes degradation of at least a portion of the matrix.
